SEQUENCE LISTING (22 sequences)

SEQ ID NO: 1 - protein, 37 aa, Synechocystis PCC6803
MVKVIGRRSLGVQRIFDIGLPQDHNFLLANGAIAANC

SEQ ID NO: 2 - protein, 112 aa, Synechocystis PCC6803
AEYCLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDN

SEQ ID NO: 3 - protein, 36 aa, Nostoc sp. PCC73102
MIKIATRKYLGKQNVYDIGVERDHNFALKNGFIASN

SEQ ID NO: 4 - protein, 102 aa, Nostoc sp. PCC73102
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 5 - protein, 35 aa, Artificial Sequence
  Amino acid sequence for Cfa DnaE C-terminus intein domain
VKIISRKSLGTQNVYDIGVEKDHNFLLKNGLVASN

SEQ ID NO: 6 - protein, 100 aa, Artificial Sequence
  Amino acid sequence for Cfa DnaE N-terminus intein domain
CLSYDTEILTVEYGFLPIGKIVEERIECTVYTVDKNGFVYTQPIAQWHNRGEQEVFEYCLEDGSIRATKDHKFMTTDGQMLPIDEIFERGLDLKQVDGLP

SEQ ID NO: 7 - protein, 38 aa, Artificial Sequence
  Amino acid sequence for gp41-1 C-terminus intein domain
MMLKKILKIEELDERELIDIEVSGNHLFYANDILTHNS

SEQ ID NO: 8 - protein, 88 aa, Artificial Sequence
  gp41-1 N-terminus intein domain
CLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 9 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 10 - protein, 56 aa, Homo sapiens
NPFSTQDTDLDLEMLAPYIPMDDDFQLRSFDQLSPLESSSASPESASPQSTVTVFQ

SEQ ID NO: 11 - protein, 11 aa, Artificial Sequence
  SsrA tag
AANDENYALAA

SEQ ID NO: 12 - protein, 150 aa, Artificial Sequence
  Wherein XXXXXX is the extein and cyclic peptide to be produced; the first X is C, S, or T, and subsequent Xs are shown as tilde in the description, which denotes an amino acid of the cyclic peptide sequence
  VARIANT (43)..(43): Xaa is Cys, Ser, or Thr
  VARIANT (44)..(48): Xaa is, each independently, any amino acid or absent
HHHHHHMIKIATRKYLGKQNVYDIGVERYHNFALKNGFIASNXXXXXXCLSYDTEILTVEYGILPIGKIVEKRIECTVYSVDNNGNIYTQPVAQWHDRGEQEVFEYCLEDGCLIRATKDHKFMTVDGQMMPIDEIFERELDLMRVDNLPN

SEQ ID NO: 13 - protein, 6 aa, Artificial Sequence
  Hexahistidine (6xHis) tag
HHHHHH

SEQ ID NO: 14 - protein, 36 aa, Artificial Sequence
  Constituent comprising the C-terminus intein domain
MIKIATRKYLGKQNVYDIGVERYHNFALKNGFIASN

SEQ ID NO: 15 - protein, 102 aa, Artificial Sequence
  Constituent comprising the N-terminus intein domain
CLSYDTEILTVEYGILPIGKIVEKRIECTVYSVDNNGNIYTQPVAQWHDRGEQEVFEYCLEDGCLIRATKDHKFMTVDGQMMPIDEIFERELDLMRVDNLPN

SEQ ID NO: 16 - protein, 206 aa, Artificial Sequence
  Wherein XXXXXX is the extein and cyclic peptide to be produced; the first X is C, S, or T, and subsequent Xs are shown as tilde in the description, which denotes an amino acid of the cyclic peptide sequence
  VARIANT (43)..(43): Xaa is Cys, Ser, or Thr
  VARIANT (44)..(48): Xaa is, each independently, any amino acid or absent
HHHHHHMIKIATRKYLGKQNVYDIGVERYHNFALKNGFIASNXXXXXXCLSYDTEILTVEYGILPIGKIVEKRIECTVYSVDNNGNIYTQPVAQWHDRGEQEVFEYCLEDGCLIRATKDHKFMTVDGQMMPIDEIFERELDLMRVDNLPNNPFSTQDTDLDLEMLAPYIPMDDDFQLRSFDQLSPLESSSASPESASPQSTVTVFQ

SEQ ID NO: 17 - protein, 56 aa, Artificial Sequence; identical to SEQ ID NO: 10
  Comprising amino acids 548-603 of the full length ODD domain of HIF-1alpha

SEQ ID NO: 18 - protein, 46 aa, Artificial Sequence
  C-terminus intein domain (with N-terminus 6xHis tag)
MGHHHHHHGSGVKIISRKSLGTQNVYDIGVGEPHNFLLKNGLVASN

SEQ ID NO: 19 - protein, 101 aa, Artificial Sequence
  N-terminus intein domain
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 20 - protein, 280 aa, Artificial Sequence
  Wildtype Cfa splice junctions (CFN and AEY) incorporated
CFNWSHPQFEKGGGSGGGSGGSAWSHPQFEKGGSGGEFMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLKERVTAAGITLGMDELYKAEY

SEQ ID NO: 21 - protein, 56 aa, Artificial Sequence; identical to SEQ ID NO: 10
  oxygen dependent degradation (ODD) domain from HIF-1alpha, comprising the key residue P564 for hydroxylation, incorporated C-terminus to N-terminus intein domain

SEQ ID NO: 22 - protein, 246 aa, Artificial Sequence
  fluorescent protein mCherry fused C-terminal to ODD domain, followed by a FLAG tag
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKTGDYKDDDDK

(19) United States
(12) Patent Application Publication
(10) Pub. No.: US 2024/0279646 A1
(43) Pub. Date: Aug. 22, 2024

(54) PEPTIDE

(71) Applicant: University of Southampton, Southampton Hampshire (GB)

(72) Inventor: Ali TAVASSOLI, Southampton Hampshire (GB)

(21) Appl. No.: 17/787,061

(22) PCT Filed: Dec. 17, 2020
(86) PCT No.: PCT/GB2020/053263

§ 371 Date: Jun. 17, 2022

(30) Foreign Application Priority Data
Dec. 18, 2019 (GB) 1918693.1

(51) Int. Cl. C12N 15/10; C07K 7/64; C12N 15/85

(52) U.S. Cl. CPC C12N 15/1093; C07K 7/64; C12N 15/85

(57) ABSTRACT

The invention provides a non-toxic method for producing cyclic peptides within a mammalian cell, comprising the steps of a) introducing a vector into the mammalian cell, wherein the vector comprises a construct encoding a C-terminus intein domain, a polypeptide sequence to be cyclised, an N-terminus intein domain, and a degradation tag, wherein the degradation tag is attached to at least one intein domain, and b) expressing the construct to produce an intermediate comprising an active intein and the polypeptide sequence, wherein the active intein, once formed, undergoes splicing and cyclises the polypeptide and wherein the degradation tag degrades the active intein. The invention further provides a cyclic peptide library produced according to this method, and the incorporation of the non-toxic cyclic peptide-producing construct of the above method into a genetic construct, a vector, or a mammalian cell.

This application is a national phase application under 35 U.S.C. § 371 of International Application No. PCT/GB2020/053263, filed Dec. 17, 2020, which claims the benefit of Great Britain Patent Application No. 1918693.1, filed Dec. 18, 2019, the entirety of each of which is incorporated herein by reference.

A sequence listing is submitted herewith as an ASCII compliant text file named “EPCLP0103US_ST25.txt”, created on Dec. 27, 2022 and having a size of 26,342 bytes. The content of the aforementioned file is hereby incorporated by reference in its entirety.

### FIELD OF INVENTION

The present invention relates to the non-toxic production of cyclic peptides via modifications to a split-intein circular ligation of peptides and proteins (SICLOPPS) methodology for enhanced efficiency within mammalian cells.

### BACKGROUND

The use of cyclic peptides in the early stages of drug discovery has become increasingly prevalent within pharmaceutical research and development. These polypeptides, ranging in length from just two conjoined amino acids to peptides comprising hundreds of such residues, are particularly useful for identifying protein-protein interaction inhibitors, with a further use in serving as key starting points for the design of drug-like small molecules.

Peptides have a particular utility as ligands against otherwise “undruggable” targets. Such undruggable targets may be intracellular molecules, specific protein-protein interactions, and are generally unsuitable to small molecules and biologics. The further cyclisation, or ring closure, of peptides enhances the lifespan of such molecules in vivo with a subsequent marked improvement in their pharmacokinetic dynamics. Whilst the scope of useful cyclic peptides found in nature is somewhat limited, the production of synthetic polypeptides as such in the laboratory opens an avenue of potential for discovering candidate drugs.

As a result, the generation of large scale libraries of synthetic cyclic peptides has become a cornerstone of modern drug discovery with substantial economic and commercial implications. Such genetically-encoded libraries allow for high-throughput screening and rapid deconvolution of hits that bind to target proteins, by sequencing DNA or mRNAtags that may be bound to each peptide.

One increasingly used method for intracellular cyclic peptide library generation is termed split-intein circular ligation of peptides and proteins (SICLOPPS). This readily accessible method can generate libraries of over 100 million members with significant speed and simplicity, with its intracellular nature allowing for integrated functional assays in vivo.

The approach makes use of intein splicing to cyclise each peptide of interest, or “extein”. Inteins are unique autoprocessing protein domains that can undergo a self-excising event from a larger precursor polypeptide through the cleavage of two peptide bonds, whilst ligating the N- and C-termini of the flanking extein sequences with a new peptide bond. A “split-intein”, more specifically, has its polypeptide sequence originating from two genes and can result in the flanking of an extein by two separate N-intein and C-intein domains. Following translation, the two domains non-covalently reassemble into a canonical active intein to carry out protein splicing.

A SICLOPPS construct encodes a C-terminus intein domain followed by the extein polypeptide sequence to be cyclised, and an N-terminus intein domain. Upon transcription and translation, the flanking regions associate to give an active intein that self-excises and cyclises the remaining polypeptide sequence between the C- and N-terminus intein domains as a result of splicing. Peptides of varying length and amino acid composition can be incorporated into the SICLOPPS method, providing that the first amino acid of the target peptide is a nucleophilic cysteine, serine or threonine.

The technique provides a simple method for generating cyclic peptide libraries, requiring just a SICLOPPS plasmid, a degenerate oligonucleotide, and a handful of straightforward molecular biology steps. The degenerate oligonucleotide will have been designed to determine the ring size of the cyclic peptides, the number of randomised amino acids, and any set amino acids to be incorporated. Each oligonucleotide, containing a unique extein sequence of interest, is integrated into a SICLOPPS plasmid via PCR-digest and ligation techniques to create a library. The plasmid library can then be transformed into cells containing a phenotypic assay, for example, and then screened. The identity of the active cyclic peptides is revealed by isolating the SICLOPPS plasmids from cells that show the desired phenotype, followed by DNA sequencing (Tavassoli 2017, Curr Opin Chem Biol 38: 30-35).

The advent of SICLOPPS introduced the benefit of interfacing cyclic peptide libraries with assays in a variety of organisms: ranging from E. coli, yeast, and mammalian cells. Intracellular functional assays can be conducted against a variety of targets, thus not only assessing affinity of each member of the library, but also its function against the given target. SICLOPPS libraries are DNA-encoded, which gives a large amount of control over the makeup of the library and allows a variety of libraries to be easily produced and screened against such targets. Examples of variations in SICLOPPS libraries that are easy to implement include: cyclic peptides of different ring sizes, libraries with different amino acid composition, or inclusion of a given amino acid, or motif in a set position in every member of the library. Thus, the user has absolute control over the makeup of their cyclic peptide library via the degenerate oligonucleotide that encodes it.

SICLOPPS originally used the DnaE split-inteins from Synechocystis sp. PCC6803 termed “Ssp” inteins. Ssp inteins have a relatively slow splice rate and a significant sensitivity to amino acid changes near the splice junctions, meaning that a significant portion of the cyclic peptide library may not actually be cyclic peptides, but rather exist as the partially spliced intein. Such limitations of the technique were, however, overcome with the adaptation of faster splicing and more promiscuous “Npu” inteins engineered from Nostoc punctiforme.

Yet despite the apparent progress from using alternative intein types, the use of inteins themselves still pose an issue with regards to low efficiency levels of the technique in cells. Townend and Tavassoli (2016) investigated the effects of SICLOPPS-generated cyclic peptide libraries on cell viability in E. coli (Townend & Tavassoli 2016, ACS Chem Biol 11(6): 1624-1630). The data demonstrated that, despite their rapid rate of splicing and tolerance to variation in extein sequence, some 42% of an Npu SICLOPPS library was found to be cytotoxic to their E. coli host, significantly reducing their utility. Owing to the study's utilisation of three different E. coli strains (DH5a, BW27786, and BL21), it was deemed unlikely that such observed effects would be strain specific. The assay was further carried out with the less favourable Ssp inteins, with results indicating that around 14% of the library members also affected host viability. While it is likely that a portion of the cyclic peptides encoded by the library are inherently toxic to E. coli, for example through interference with a critical protein or pathway, both sets of inteins encoded the same library in the experiment. The higher level of toxicity observed with the Npu inteins, thus, can only be attributed to the Npu inteins themselves.

In a 2016 study, scientists engineered an SsrA tag (AANDENYALAA; SEQ ID NO: 11) to the C-terminus of a protein of interest, in order to target the spliced inteins for intracellular degradation (Townend & Tavassoli 2016, ACS Chem Biol 11(6): 1624-1630). Addition of the SsrA sequence was shown to direct the tagged protein to the ClpXP machinery native to E. coli for degradation, reducing the half-life of the tagged protein to around 5 minutes.

Whilst there has been an investigation into reducing the cytotoxicity of Npu SICLOPPS inteins in E. coli, there exists no such described alternative approach applicable for use in mammalian cells that differ in cellular functions to prokaryotic cells. With an ever increasing demand for functional assays of cyclic peptides in mammalian cells for drug discovery, a modification of SICLOPPS to improve the efficiency would help elucidate compounds against mammalian-specific targets.

Thus, there exists a need for a modified SICLOPPS approach that generates cyclic peptides in mammalian cells in a more efficient manner.

Kinsella et al 2002 JBC 277: 37512-37518 describes the production of cyclic peptide libraries with up to 160,000 members in mammalian cells using Ssp SICLOPPS inteins with no mention of, or investigation into toxicity associated with the intein.

### SUMMARY OF INVENTION

The toxicity of inteins in mammalian cells was not investigated by Kinsella et al. The inventors have surprisingly found that mammalian cells are also susceptible to toxicity arising from active inteins. Prior to this, the problem of intein associated toxicity in mammalian cells was not recognised. The inventors have devised a degradation tag system suitable for use in mammalian cells and which obviates the intein-associated toxicity allowing the split intein system to be widely used in mammalian cells for the production of cyclic peptides.

This invention is based on the surprising discovery that it is possible to alter a mammalian cell-based SICLOPPS methodology to include intein-attached degradation tags in order to minimise any resultant intein-induced cytotoxicity. The attachment of a degradation tag to either the N-terminus or C-terminus intein domain will allow the canonical active intein, following splicing and cyclisation of the extein of interest, to be directed for degradation via the mammalian cell's degradation pathway. The approach therefore prevents a detrimental accumulation of cytotoxic inteins from being formed during cyclic peptide production within mammalian cells. Such degradation-tagged inteins can therefore be used in a modified SICLOPPS methodology to produce a cyclic peptide library within mammalian cells with greater efficiency. Importantly, the intein is able to splice before it is degraded.

Thus in a first aspect of the invention, there is provided a method for the non-toxic production of a cyclic peptide in a mammalian cell comprising: a) introducing a vector into a mammalian cell, wherein the vector comprises a construct encoding a C-terminus intein domain, a polypeptide sequence to be cyclised, an N-terminus intein domain, and a degradation tag, wherein the degradation tag is attached to at least one intein domain; and b) expressing the construct to produce an intermediate comprising an active intein and the polypeptide sequence, wherein the active intein, once formed, undergoes splicing and cyclises the polypeptide and wherein the degradation tag degrades the active intein.

The invention also provides a mammalian cell produced by the method of the first aspect of the invention. For example, the invention provides a cell expressing a cyclic peptide wherein the mammalian cell is produced by a method comprising:
a) introducing a vector into the mammalian cell, wherein the vector comprises a construct encoding a C-terminus intein domain and a N-terminus intein domain of a split intein; a polypeptide sequence to be cyclised; and a degradation tag, wherein the degradation tag is attached to at least one intein domain;
b) expressing the construct to produce an intermediate comprising an active intein and the polypeptide sequence, whereby the active intein undergoes splicing and cyclises the polypeptide and wherein the degradation tag degrades the active intein.

The invention also provides a library of mammalian cells produced according to the first aspect of the invention, i.e. the library comprises a number of mammalian cells each comprising a different nucleic acid that encodes a different cyclic peptide, where the nucleic acid is a nucleic acid of the invention as described herein. In some embodiments the library of mammalian cells produced by the method according to the first aspect of the invention comprises at least 128,000 at the cyclic peptide level, optionally for example at least 150,000 or at least 200,000 members at the cyclic peptide level. As the skilled person will appreciate, it is possible to produce a library comprising millions of different genetic constructs, but if the protein or peptide (in this case the cyclic peptide) that the gene encodes is toxic to the cells, then those cells will be lost, reducing the number of members in the library at the protein level. For example, if a library of 2 million members is produced at the genetic level, but the active intein is toxic, only for example 1 million members expressing cyclic peptides would be obtained. Since the present invention addresses the toxicity associated with inteins it is possible to produce much larger cyclic peptide libraries, or much larger libraries of mammalian cells produced according to the method of the first aspect of the invention.

In some embodiments the library of mammalian cells produced by the method according to the first aspect of the invention comprises at least 128,000 members, for example at least 130,000, 150,000, 200,000, 300,000, 400,000, 500,000, 600,000, 700,000, 800,000, 900,000, 1 million, 1.5 million, 2 million, 2.5 million, 3 million, 3.2 million, 3.5 million or at least 4 million members at the protein level.

By at the protein level, we include the meaning of the level of the expressed cyclized peptide level.

It will be clear that whilst the use of split inteins to produce a cyclic peptide in a mammalian cell as reported in the art will result in a mammalian cell that comprises active inteins, that may be toxic, the cells of the present invention due to the use of the degradation tag comprise no active inteins, or substantially no active inteins, for example no or substantially no toxic active inteins.

The invention also provides a cell lysate prepared from a mammalian cell of the invention.

In a second aspect of the invention, there is provided a cyclic peptide library produced by the method according to the first aspect of the invention.

In a third aspect of the invention, there is provided a genetic construct comprising a polynucleotide cassette encoding a C-terminus intein domain, a polypeptide sequence to be cyclised, an N-terminus intein domain, and a degradation tag suitable for use in mammalian cells, wherein the degradation tag is attached to at least on intein domain and wherein, once expressed, an active intein is formed.

In a fourth aspect of the invention, there is provided a vector comprising the genetic construct according to the third aspect of the invention.

In a fifth aspect of the invention, there is provided a mammalian cell comprising the vector according to the fourth aspect of the invention or the genetic construct according to the third aspect of the invention. The invention also provides a library of mammalian cells comprising the vector according to the fourth aspect of the invention or the genetic construct according to the third aspect of the invention. In some embodiments the library of mammalian cells comprises at least 200,000 members, for example at least 300,000, 400,000, 500,000, 600,000, 700,000, 800,000, 900,000, 1 million, 1.5 million, 2 million, 2.5 million, 3 million, 3.2 million, 3.5 million or at least 4 million members.

In a sixth aspect of the invention, there is provided a method of producing a cyclic library according to the method of the first aspect of the invention.

### DESCRIPTION OF FIGURES

The invention is illustrated with reference to the following drawings, in which:

FIG. 1 shows the initiation of the mechanism of SICLOPPS, in which the N-terminus and C-terminus intein domains, flanking an extein peptide sequence of interest, non-covalently associate to form a canonical active intein (adapted from Townend & Tavassoli 2016, ACS Chem Biol 11(6): 1624-1630).

FIG. 2 shows the mechanism of SICLOPPS following the formation of an active intein. In a three step process involving a thioester intermediate and a lariat intermediate, the active intein splices to cyclise the target peptide extein.

FIG. 3 shows how a cyclic peptide library can be generated from a SICLOPPS plasmid library. Plasmids containing appropriate origins of replication, selectable markers and promoters, and a SICLOPPS construct of interest, are transfected into mammalian cells. Following transcription and translation, the expressed inteins, in this case DnaE inteins, cyclise each peptide of interest to produce an intracellular library of such molecules.

FIG. 4 shows a SICLOPPS construct for an eGFP/YFP peptide designed with the addition of a degradation tag to the N-intein. The degradation tag attached is the oxygen dependent degradation (ODD) domain of the hypoxia-inducible factor-1 alpha (HIF-1α) subunit. Further additions to the construct include affinity tags, a fluorescent tag of mCherry, and a FLAG tag for antibody recognition.

FIG. 5 shows a fluorescent microscopy image of a SICLOPPS plasmid according to FIG. 4 that had been transfected into Hela cells which were subsequently placed in the presence of oxygen with or without 100 UM of deferoxamine (DFX) treatment. The inteins should degrade only in the presence of oxygen and absence of DFX. The results show that the inteins, associated with mCherry fluorescence, degraded in normoxia in comparison to DFX experiments which displayed mCherry fluorescence. The extein, marked with GFP, remained present under both conditions.

FIG. 6 shows the fluorescent microscopy image of a SICLOPPS plasmid according to FIG. 4, further containing a P564G mutation in the degradation tag, that had been transfected into Hela cells which were subsequently placed in the presence of oxygen with or without 100 UM of deferoxamine (DFX) treatment. In this mutated construct, the inteins should never be degraded regardless of the condition, as was observed in the results.

FIG. 7 shows a western blot analysis for the wildtype (WT) and P564G mutant SICLOPPS plasmids as described above, which had been transfected into Hela cells and incubated under normoxia, hypoxia, or DFX-treated conditions. The hypoxia and DFX conditions showed no intein degradation for the wildtype plasmid, whilst the inteins were degraded in normal oxygen conditions.

FIG. 8 shows the cell counts over 48 hours for Hela cells transfected with the WT and P564G mutant SICLOPPS plasmids as described above, under normoxia or hypoxia. The trend shows that under hypoxia, without the inteins being degraded, a decrease in cell number due to cytotoxicity occurs for both the WT and P564G SICLOPPS plasmids over time. Under normal conditions however, the cell number of WT-transfected cells, with degradation of inteins, is maintained over time, relative to the proline-mutated intein-containing cells which decrease in number due to cytotoxicity.

FIG. 9 shows Trex293 cells transfected with plasmids encoding GFP-Npu-ODDD-mCherry. Splicing (WT) or non-splicing (C1A). Gates represent Q1: mCherry+GFP−, Q2: mCherry+GFP+, Q3: mCherry− GFP+, Q4: mCherry− GFP−. A) GFP-WT without DFX. Q1: 2.53%, Q2: 47.5%, Q3: 32.1%, Q4: 17.8%. B) GFP-WT with DFX Q1: 3.24%, Q2: 69.3%, Q3: 10.8%, Q4: 16.6%. C) GFP-C1A without DFX Q1: 40.4%, Q2: 37.5%, Q3: 0.65%, Q4: 21.4%. D) GFP-C1A with DFX Q1: 25.0%, Q2: 54.3%, Q3: 0.18%, Q4: 20.5%. E) Overlay of mCherry+ cells from A (dark grey; −DFX) and B (light grey; +DFX): addition of DFX prevents mCherry (i.e the inteins) degradation. Value of mCherry-A median A: 265 units, B: 618 units.

FIG. 10 Trex293 cells integrated with plasmids encoding GFP-Npu-ODDD-mCherry splicing (WT) Gates represent Q1: mCherry+GFP−, Q2: mCherry+GFP+, Q3: mCherry− GFP+, Q4: mCherry− GFP−. A) GFP-WT without DFX. Q1: 0.035%, Q2: 0.84%, Q3: 67.4%, Q4: 31.7%. B) GFP-WT with DFX Q1: 0.17%, Q2: 35.1%, Q3: 29.4%, Q4: 35.4%.

FIG. 11 Cells without DFX (−dfx) and cells with DFX (+dfx) were assessed for viability after 24h of incubation. Values are triplicate (+/−SD). Viability was normalized for each cell line to their −dfx control.

FIG. 12 Plasmid map for GFP-Npu-mCherry-ODDD used in examples 5 and 6.

### DESCRIPTION OF SEQUENCE LISTING

SEQ ID NO: 1 is an amino acid sequence for the C-terminus intein domain of an Ssp DnaE split intein, isolated from Synechocytis sp. strain PCC6803.
SEQ ID NO: 2 is an amino acid sequence for the corresponding N-terminus intein domain of an Ssp DnaE split intein, isolated from Synechocytis sp. strain PCC6803.
SEQ ID NO: 3 is an amino acid sequence for the C-terminus intein domain of an Npu DnaE split intein, isolated from Nostoc sp. strain PCC73102.
SEQ ID NO: 4 is an amino acid sequence for the corresponding N-terminus intein domain of an Npu DnaE split intein, isolated from Nostoc sp. Strain PCC73102.
SEQ ID NO: 5 is an amino acid sequence for the C-terminus intein domain of an artificial Cfa DnaE split intein engineered for enhanced stability and activity.
SEQ ID NO: 6 is an amino acid sequence for the corresponding N-terminus intein domain of an artificial Cfa DnaE split intein engineered for enhanced stability and activity.
SEQ ID NO: 7 is an amino acid sequence for the C-terminus intein domain of an “ultrafast” gp41-1 DnaE split intein engineered for very fast splicing activity.
SEQ ID NO: 8 is an amino acid sequence for the corresponding N-terminus intein domain of an “ultrafast” gp41-1 DnaE split intein engineered for very fast splicing activity.
SEQ ID NO: 9 is an amino acid sequence for Homo sapiens hypoxia-inducible factor-1 alpha (HIF-1α) subunit comprising the oxygen dependent degradation (ODD) domain, which may be used as a degradation tag.
SEQ ID NO: 10 is an amino acid sequence for the oxygen dependent degradation (ODD) domain of Homo sapiens hypoxia-inducible factor-1 alpha (HIF-1α) subunit, which may be used as a degradation tag.

### DETAILED DESCRIPTION

The invention is predicated on the surprising discovery that the addition of a degradation tag to a SICLOPPS-based methodology allows for the production of cyclic peptides within mammalian cells without generating an accumulation of cytotoxic intein by-products. Cyclic peptides can thus be produced in mammalian cells without the low efficiency levels normally associated with such a method.

The term “cyclic peptide” as used herein refers to a polypeptide or protein that has been “cyclised”, in which its constituent atoms form a ring. For example, a linear peptide is cyclised when its free amino (N)-terminus is covalently bonded to its free carboxy (C)-terminus, i.e. in a head to tail format, such that no free C- or N-termini remain in the peptide. As referenced herein, the terms “peptide” and “polypeptide” can be used interchangeably.

The invention provides a method of producing cyclic peptides within mammalian cells by an altered SICLOPPS methodology to incorporate the use of degradation tags.

The term “mammalian cell” refers to a eukaryotic cell with structurally defined intracellular organisation, as opposed to bacteria and archaea. Mammalian cells are often used in cell culture, for example the use of Chinese Hamster Ovary (CHO) cells, the most common mammalian cell line used for mass production of therapeutic proteins (Wurm 2004, Nat Biotech 22(11): 1393-1398).

SICLOPPS or the “split-intein circular ligation of peptides and proteins” takes advantage of intein splicing for the generation of a cyclic peptide.

As used herein, the word “intein” means a naturally-occurring or artificially constructed polypeptide sequence embedded within a precursor protein that can catalyse a splicing reaction during post-translation processing of the protein. An intein can excise itself from the precursor protein and join the remaining portions with a peptide bond in a process named “splicing”. A “split-intein” is an intein that has two or more separate components not fused to one another, encoded by two separate genes. In some cases, the split-intein components will flank a polypeptide sequence therein referred to as an “extein”. When flanking an extein, the split-intein components are referred to as an N-terminus and C-terminus intein domain in respect to the N—and C-termini of the extein.

In some embodiments of all aspects of the invention the intein is an intein that is toxic to mammalian cells. By toxic we include the meaning that the intein negatively affects the growth rate of the mammalian cells, or causes apoptosis or necrosis.

The mammalian cells can be any mammalian cells in which it is desirous to express a cyclic peptide.

More than 350 types of inteins are recognised at present, each or which can have differing rates of catalysing a splicing reaction. The nomenclature of inteins is based on the scientific name of the organism to which it is found. Ssp inteins, for example, were first isolated from Synechocytis spp, whilst faster splicing Npu inteins were first isolated from Nostoc punctiforme. A database comprising a list of some of the known inteins can be found at http://www.biocenter.helsinki.fi/bi/iwai/InBase/tools.neb.com/inbase/list.html.

The intein used in the invention may be any intein that would splice faster than its degradation time by an attached degradation tag. The skilled person can select an appropriate intein for use with a corresponding degradation tag.

In one embodiment the intein may be a Cfa intein. In a preferred embodiment the intein may be a split Cfa intein comprising the amino acid sequences of SEQ ID NO: 5 and SEQ ID NO: 6 for the C-terminus and N-terminus intein domains, respectively.

In another preferred embodiment the intein may be an Ssp intein. In a further preferred embodiment the intein may be a split Ssp intein comprising the amino acid sequences of SEQ ID NO: 1 and SEQ ID NO: 2 for the C-terminus and N-terminus intein domains, respectively.

In another preferred embodiment the intein may be a gp41-1 intein. In a further preferred embodiment the intein may be a split gp41-1 intein comprising the amino acid sequences of SEQ ID NO: 7 and SEQ ID NO: 8 for the C-terminus and N-terminus intein domains, respectively.

In yet another preferred embodiment the intein may be an Npu intein. In a most preferred embodiment the intein may be a split Npu intein comprising the amino acid sequences of SEQ ID NO: 3 and SEQ ID NO: 4 for the C-terminus and N-terminus intein domains, respectively.

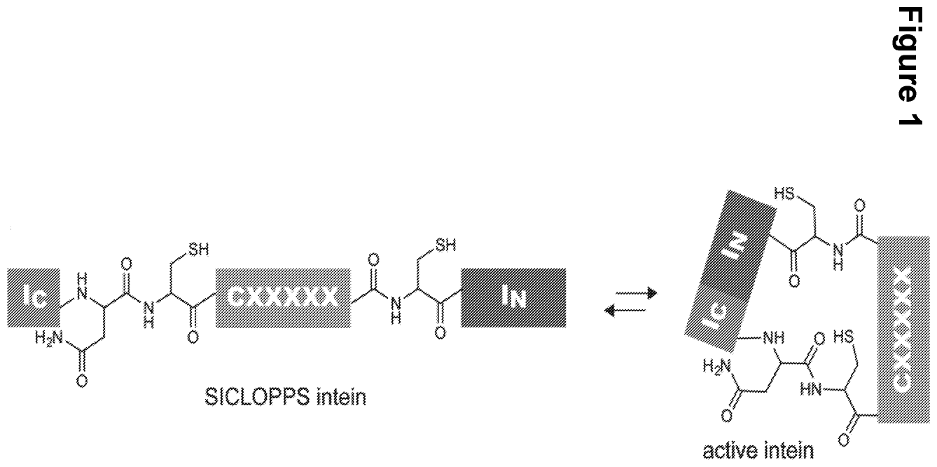

The process of SICLOPPS is known in the art, as referenced by Tavassoli 2017, Curr Opin Chem Biol 38: 30-35. A SICLOPPS method makes use of a construct containing a C-terminus intein domain followed by an extein polypeptide sequence to be cyclised, and an N-terminus intein domain. The construct is arranged as such so that, following translation of the mRNA sequence, the N- and C-terminus intein domains flanking the intervening extein are capable of non-covalently associating to form a functional active intein (FIG. 1) that subsequently catalyses the splicing reaction that produces the cyclic polypeptide.

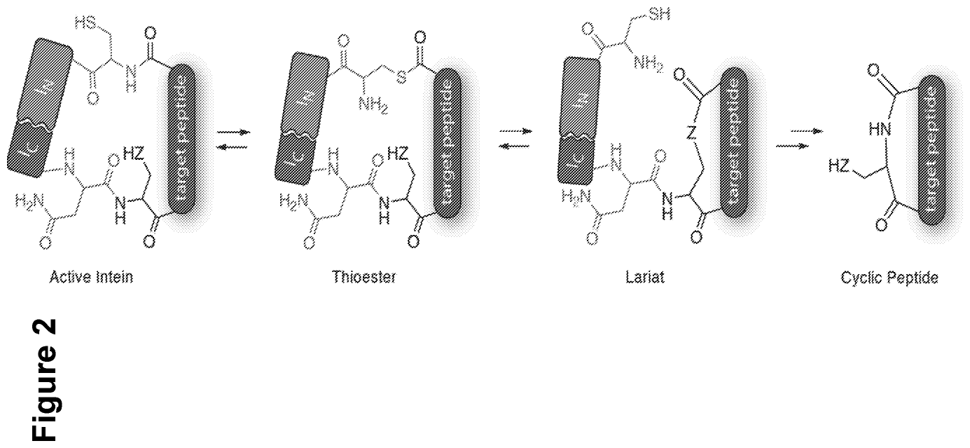

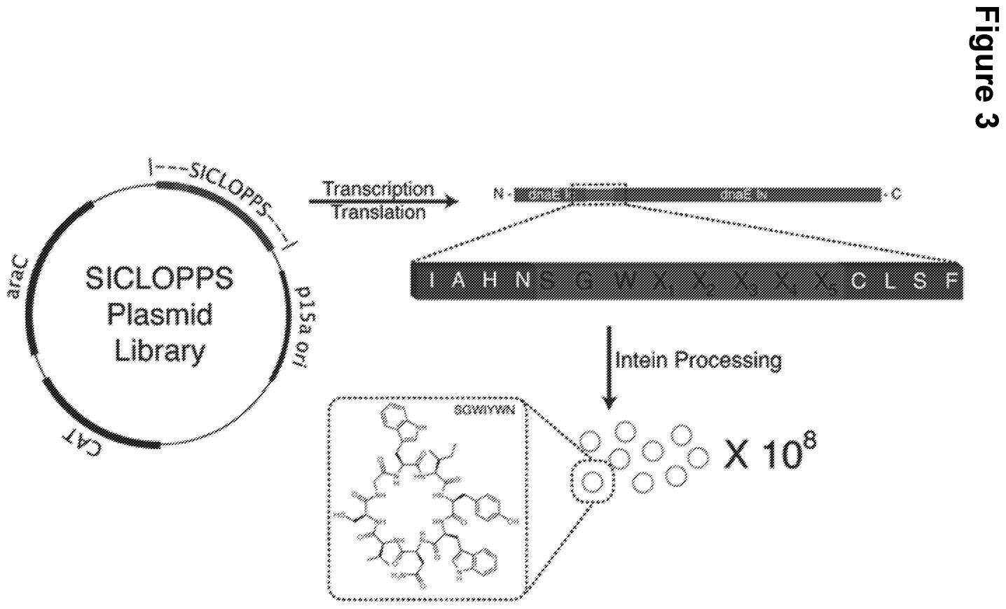

As shown in FIG. 2, the folded SICLOPPS construct, or “fusion protein”, with its active canonical intein, will catalyse an N-to-S acyl shift at the N-terminus intein domain and extein junction to produce a thioester intermediate. The thioester intermediate will undergo transesterification with a side-chain nucleophile at the opposite C-terminus intein domain and extein junction to form a lariat intermediate. Finally, an asparagine side-chain cyclisation and subsequent X-N acyl shift liberates the cyclic peptide from the intein (Scott et al., 1999, PNAS 96(24): 13638-13643).

Such a method thus results in the production of a cyclic peptide and a bi-product comprising the now unneeded intein polypeptide.

Thus in some embodiments of the invention there is an altered SICLOPPS method that uses a polypeptide construct comprising an Npu split intein that may comprise the following sequence:

HHHHHHMIKIATRKYLGKQNVYDIGVERYHNFALKNGFIASN
X~~~~~CLSYDTEILTVEYGILPIGKIVEKRIECTVYSVDNNGNIYTQPVAQWHDR
GEQEVFEYCLEDGCLIRATKDHKFMTVDGQMMPIDEIFERELDLMRVDNLPN
(SEQ ID NO: 12; wherein ″~″ is represented as further Xs).

Wherein X ˜˜˜˜˜ is the extein and cyclic peptide to be produced; X is C, S, or T, and “˜” denotes an amino acid of the cyclic peptide sequence. It is necessary for the functioning of the splicing that the first position may be occupied by an invariant cysteine, serine, or threonine residue. It will be apparent to one skilled in the art that any sequence may be inserted after “X” in the sequence above. The sequence may be one or more amino acids in length.

In a preferred embodiment the sequence may be three or more amino acids in length. In a most preferred embodiment the sequence may be at least six amino acids in length.

The above sequence comprises the following constituents:

1.
(SEQ ID NO: 13)
HHHHHH

An optional hexahistidine (6xHis) tag to assist in purification, or any other such affinity tag, may be included within the construct. Thus in one embodiment the base SICLOPPS construct will further include an affinity tag. In a preferred embodiment, the construct will include a 6xHis tag. In yet another preferred embodiment, the construct will include a 2xStrep tag.

2.
(SEQ ID NO: 14)
MIKIATRKYLGKQNVYDIGVERYHNFALKNGFIASN

Comprising the C-terminus intein domain.

3.
X~~~~~
Comprising the polypeptide extein sequence to be cyclised.
4.
(SEQ ID NO: 15)
CLSYDTEILTVEYGILPIGKIVEKRIECTVYSVDNNGNIYTQPVAQWHDRGEQE
FEYCLEDGCLIRATKDHKFMTVDGQMMPIDEIFERELDLMRVDNLPN

Comprising the N-terminus intein domain.

In some embodiments the SICLOPPS construct may be further modified to include a tag for antibody recognition. In a preferred embodiment, the tag for antibody recognition may be a FLAG tag.

The invention provides an altered SICLOPPS method wherein the SICLOPPS construct, as exemplified above, is modified with the addition of a degradation tag, suitable for use in mammalian cells, attached to either of the N-terminus or C-terminus intein domains.

The term “degradation tag” is intended to encompass peptide sequences that mark a protein for degradation by a cell's degradation machinery. In mammalian cells, a major pathway of selective protein degradation is the ubiquitin-proteasome pathway. Ubiquitin-dependent protein degradation has a natural role in many biological processes, including signal transduction, cell cycle progression, and transcriptional regulation (Groulx & Lee 2002, Mol Cell Biol 22(15): 5319-5336).

Ubiquitin is a small regulatory protein that can be added to a substrate protein in a process called ubiquitination. The conjugation of ubiquitin is an ATP-dependent process that involves three enzymes: E1 and E2 proteins prepare the ubiquitin for conjugation; E3 ubiquitin ligases recognise the specific protein substrate to catalyse the transfer activated ubiquitin molecule. Once a protein is tagged with a single ubiquitin molecule, other E3 ubiquitin ligases are signalled to attach further ubiquitin molecules, resulting in a polyubiquitin chain attached to the substrate protein.

Proteins tagged for ubiquitination are subsequently targeted to a cellular proteasome complex wherein the ubiquitin chain is recognised by the proteasome, and the bound proteins are degraded into peptides of seven to eight amino acids long. Degradation tags can thus function by engaging the tagged protein with an E3 ubiquitin ligase resulting in the addition of a polyubiquitin chain and subsequent degradation by the proteasome.

In one embodiment of the invention there is provided an altered SICLOPPS method that uses a construct as described above which may comprise a degradation tag attached to either the N-terminus or C-terminus intein domain.

In a preferred embodiment the attached degradation tag may effect degradation at least in part by ubiquitination.

In another preferred embodiment the attached degradation tag may be a hypoxia-inducible factor-1 alpha (HIF-1α) subunit. In a further preferred embodiment the attached degradation tag may comprise the oxygen dependent degradation domain of HIF-1a that engages an ubiquitin ligase complex. In a most preferred embodiment the attached degradation tag may comprise the amino acid sequence according to SEQ ID NO: 10.

In yet another embodiment the attached degradation tag may be a tag or proteolysis targeting chimera that engages an E3 ubiquitin ligase for protein degradation.

Hypoxia-inducible factors (HIFs) are heterodimeric transcription factors comprising a constitutively expressed HIF-1B subunit and a HIF-1a subunit regulated by oxygen. HIF-1a comprises an oxygen dependent degradation (ODD) domain that contains a key proline residue P564 that is hydroxylated in normoxia to target the HIF-1a subunit for proteasomal degradation by engaging an ubiquitin ligase complex. The ubiquitin ligase complex comprises a von Hippel-Lindau tumour suppressor protein (VHL) responsible for recognising the hydroxylated P564 residue of the ODD domain of HIF-1a.

The addition of a P564-comprising sequence from the ODD domain of HIF-1a as a degradation tag to the SICLOPPS construct polypeptide thus induces degradation of the attached protein. In the present invention, upon expression of the SICLOPPS construct, the active intein, following its self-excision and splicing and cyclisation of the extein of interest, is attached from either its N-terminus or C-terminus domain to the P564-comprising polypeptide of the ODD domain. The hydroxylated P564 residue is recognised by the VHL from an ubiquitin ligase complex, and ubiquitinated and degraded by the proteasome along with the attached cytotoxic active intein.

Thus in some embodiments, the polypeptide construct as described above for use in the altered SICLOPPS methodology may further contain amino acids 548-603 of the full length ODD domain of HIF-1α, containing the key P564 for hydroxylation, attached to the N-terminus intein domain, as comprised by the following sequence:

(SEQ ID NO: 16)
HHHHHHMIKIATRKYLGKQNVYDIGVERYHNFALKNGFIASN
X~~~~CLSYDTEILTVEYGILPIGKIVEKRIECTVYSVDNNGNIYTQPVAQWHDR
GEQEVFEYCLEDGCLIRATKDHKFMTVDGQMMPIDEIFERELDLMRVDNLPNNPFS
TQDTDLDLEMLAPYIPMDDDFQLRSFDQLSPLESSSASPESASPQSTVTVFQ

Wherein:

(SEQ ID NO: 17)
NPFSTQDTDLDLEMLAPYIPMDDDFQLRSFDQLSPLESSSASPESASPQ
STVTVFQ

Comprises amino acids 548-603 of the full length ODD domain of HIF-1α.

A proteolysis targeting chimera (PROTAC) can similarly function as a degradation tag. A PROTAC is a small molecule comprising two covalently linked protein-binding domains. One domain is capable of engaging an E3 ubiquitin ligase, whilst the other binds to a target protein meant for degradation. The incorporation of a PROTAC as a degradation tag for either the N-terminus or C-terminus thus results, upon expression of the SICLOPPS construct, in the recruitment of an E3 ubiquitin ligase to the excised active intein to result in its ubiquitination and proteasomal degradation.

It is envisaged that the degradation of active inteins will enable the production of cyclic peptides within mammalian cells without an increase in cytotoxicity as shown to be associated with the active inteins.

The term “cytotoxicity” as used herein refers to a toxic quality of a compound towards cells which may result in such cells undergoing necrosis, wherein they die rapidly due to cell lysis from a loss of membrane integrity, or apoptosis, wherein the cells undergo programmed cell death. Cytotoxic effects would have an impact on the level of efficiency in utilising mammalian cells for the purposes of producing cyclic peptides, for example, as would be understood by the skilled reader.

A degradation tag may induce degradation of the protein or polypeptide it is attached to regardless of whether the attachment is direct in sequence or via a linker. Such linkers, or spacers, are short amino acid sequences that vary between 2 and 31 amino acids, implemented to separate multiple domains of a single protein.

In one embodiment of the invention there is provided an altered SICLOPPS method using a construct as described above wherein the degradation tag is attached to either the N-terminus or C-terminus intein domain either through a direct linkage or via a linker. In a preferred embodiment, the degradation tag is attached via a direct linkage.

It is envisaged that a compatible degradation tag will be incorporated into the SICLOPPS construct relative to the type of inteins used. It would be apparent to the skilled reader that the active intein need splice before it is degraded by the included degradation tag, or in other words, the degradation tag needs to induce degradation slower than the intein splices.

It is common practice within the art to optionally include a fluorescent tag within a peptide-encoding construct for experimental visualisation purposes. It is envisaged that such fluorescent tags may be attached to the degradation tags within the SICLOPPS construct; hence any degradation of the resultant expressed tagged proteins could be visualised using fluorescence microscopy or other such techniques known within the field. A wide range of fluorescent tags, or fluorophores, exist that are suitable for use in microscopy. A comprehensive list of fluorophores is available at https://www.biosyn.com.

In some embodiments of the invention there is provided an altered SICLOPPS method using a construct as described above with the addition of any fluorescent tag. In another embodiment the fluorescent tag is attached to the degradation tag incorporated within the SICLOPPS construct. In a preferred embodiment, the fluorescent tag is a DsRed fluorophore.

It is envisaged in the present method that the modified SICLOPPS construct will be introduced into mammalian cells within any suitable expression vector that can facilitate expression of the polynucleotide.

As used herein, the phrase “expression vector” means a vehicle that facilitates transcription and/or translation of a nucleic acid molecule in a suitable in vitro or in vivo system. An expression vector is “inducible” when adding an exogenous substance to a host system containing the expression vector causes the vector to be expressed, for example causing a nucleic acid molecule within the vector to be transcribed into mRNA.

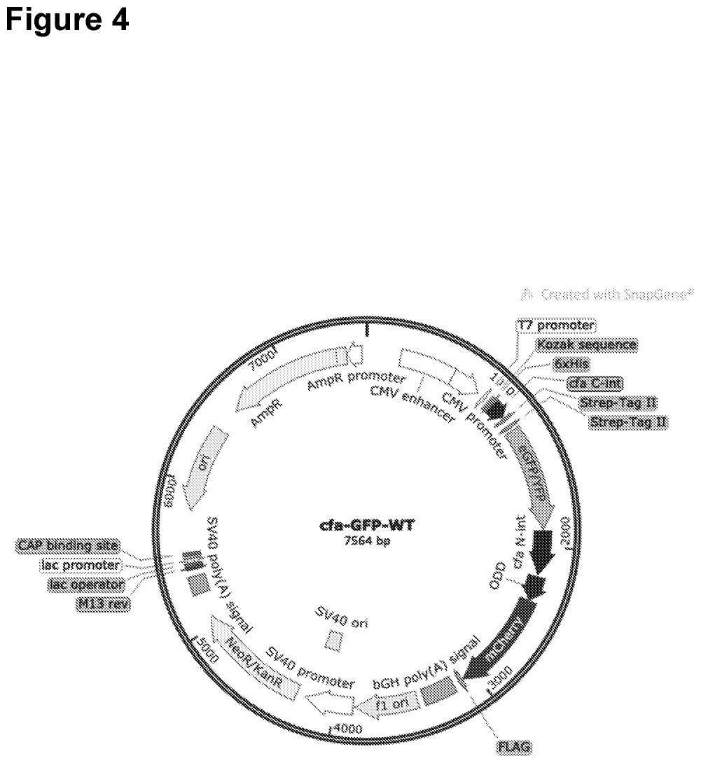

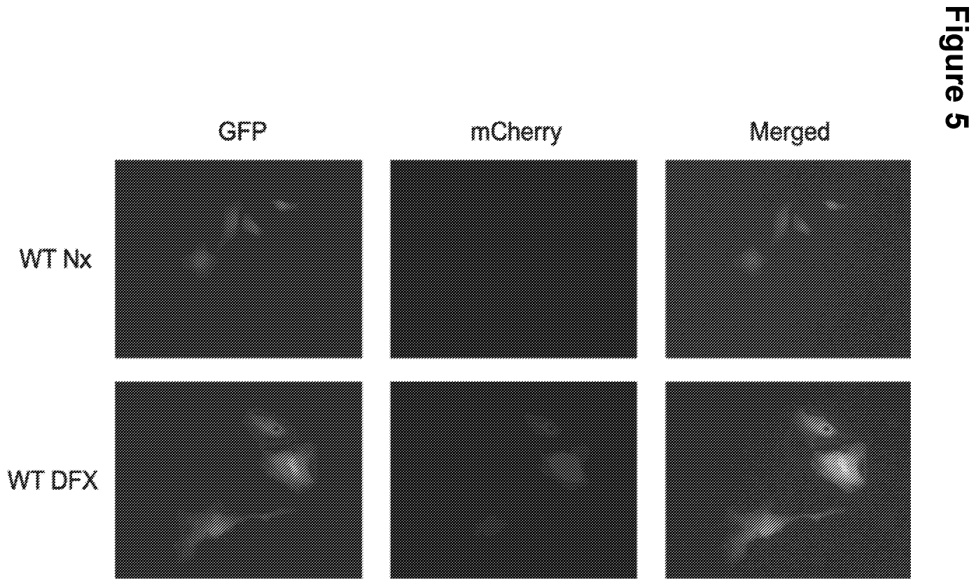

Such suitable vectors include plasmids (FIG. 4), bacteriophages, and viral vectors. A large number of these are known in the art, and many are commercially available or obtainable from the scientific community. Those of skill in the art can select suitable vectors for use in a particular application based upon, for example, the type of system selected such as mammalian cells and the expression conditions selected.

Expression vectors used within the method can include a stretch of nucleotides that encodes a target polypeptide construct and a stretch of nucleotides that operate as a regulatory domain that modulates or controls expression of nucleotide sequences within the vector. For example, the regulatory domain can be a promoter or an enhancer.

In some embodiments, the expression vectors used within the method are produced with restriction sites both between and within the nucleic acid sequences that encode the split intein portions to enable the cloning of a wide variety of cyclisation targets or splicing intermediates. In some embodiments, an expression vector of the invention can be an inducible expression vector such as an arabinose inducible vector. Such expression vectors can be generated using standard molecular biology techniques as would be known to the skilled reader. Plasmids can be transfected into mammalian cells for transient expression through several well practiced techniques within the art, such as chemical-based or electroporation-based transfection.

It is envisaged that an expression vector used within the present invention will comprise a suitable origin of replication (ORI) for use in mammalian cells. Since there are no “natural” mammalian ORIs, viral-based ORIs are often used for expression vectors intended for mammalian cells, such as viral Epstein-Barr virus (EBC) or SV40 ORIs.

Thus in one embodiment there is provided an altered SICLOPPS method that utilises a plasmid comprising a modified SICLOPPS construct as outlined above suitable for expression within mammalian cells. In a preferred embodiment the plasmid comprises a SV40 origin of replication suitable for expression within mammalian cells.

In a second aspect, the present invention provides a cyclic peptide library produced by the altered SICLOPPS method according to the first aspect of the invention.

The term “cyclic peptide library” refers to a multiple of compartmentalised cyclic peptides, often containing over 100 million peptide members.

Each member of the library may be expressed within a mammalian cell from a unique plasmid as described in the first aspect of the invention. It is envisaged that the library will contain great numbers of randomised cyclic peptides from each expressed plasmid, generated as such so that the polypeptide of interest, or extein, is randomised. The randomised polypeptide is essentially a degenerate oligonucleotide, wherein “degenerate” refers to its sequence containing a number of possible nucleotide bases. The resultant library would in theory contain a great number of possible cyclic peptide structures that may subsequently be used for pharmaceutical assays and other research purposes. The generation of cyclic peptide libraries within cells allows for functional assays to be conducted against a variety of targets.

SICLOPPS-based libraries, as outlined above, are DNA-encoded, which gives a large amount of control over the makeup of the library and allows a variety of libraries to be easily produced and screened against a given target (FIG. 3). Such variations in SICLOPPS libraries that are easy to implement include: cyclic peptides of different ring sizes; libraries with different amino acid composition, using limited codon sets; or, inclusion of a given amino acid, or motif in a set position in every member of the library. Thus, the SICLOPPS user has absolute control over the makeup of their cyclic peptide library via the degenerate oligonucleotide that encodes it.

The length of the randomised polynucleotide inserted into the vector will be dependent on various factors that may be determined by the skilled person. Of primary consideration is the size of the ultimate polypeptide expressed, and subsequent ring size of the cyclic peptide. In a preferred embodiment, the polypeptide is 6 amino acids in length. A suitable randomised polynucleotide would therefore be 18 nucleic acids in length. For cyclic peptide formation, consideration must be given to whether the length of the polypeptide is sufficient to allow the cyclisation reaction to proceed, i.e. whether the length allows a closed peptide cycle to form. In some embodiments, the peptide is cyclised by a linker of any length. Therefore, cyclic polypeptides may be achieved by encoding just two amino acids, in which case the randomised polynucleotide will be at least 6 nucleic acids in length. Another consideration is the maximum insert size tolerated by the vector and corresponding replication system.

In some embodiments, the randomised sequence may be longer, for example, at least 9, 30, 60, 90, 180, 300, 600, 900, 1,800, 3,000, or more nucleic acids in length. In preferred embodiments, the randomised nucleotide sequence is 6, 9, 12, 15, 18, 21, 24, 27, or 30 nucleotides in length. Although the randomised sequence is intended to encode a polypeptide, its length may not necessarily be a multiple of 3. For example, it may be 7, 8, 10, 11, 13, 14, 16, 17, 19, 20, 22, 23, 25, 26, 28, or 29 nucleotides in length. The randomised polynucleotide sequence may also be referred to herein as the variable sequence. In embodiments, one or more positions of the “random” or “variable” sequence may actually be fixed. For example, in such a SICLOPPS method, the first position may be occupied by an invariant cysteine, serine, or threonine residue, followed by a variable or random amino acid sequence as described within the first aspect of the invention.

In a third aspect of the invention, there is provided a genetic construct comprising a polynucleotide cassette encoding a C-terminus intein domain, a polypeptide sequence to be cyclised, an N-terminus intein domain, and a degradation tag suitable for use in mammalian cells, wherein the degradation tag is attached to at least on intein domain and wherein, once expressed, an active intein is formed.

In some embodiments, the genetic construct may further contain any of the modifications or specifications according to the first aspect of the invention.

In a fourth aspect of the invention, there is provided a vector comprising the genetic construct according to the third aspect of the invention.

In a fifth aspect of the invention, there is provided a mammalian cell comprising the vector according to the fourth aspect of the invention.

In a sixth aspect of the invention, there is provided a method of producing a cyclic library according to the method of the first aspect of the invention

In order that the invention may be more clearly understood, embodiments thereof will now be described by way of example with reference to the accompanying figures.

### Example 1

A SICLOPPS construct was designed with the addition of a degradation domain to the N-terminus intein domain, resulting in the depletion of the spliced intein product to prevent toxicity in mammalian cells. Cfa inteins were used for fast splicing and high promiscuity, containing an ERD to GEP mutation at residues 122-124 for increased amino acid tolerance at the +2 residue. The construct was designed with the following sequence:

C-terminus intein domain (+N-terminus 6xHis tag):
(SEQ ID NO: 18)
MGHHHHHHGSGVKIISRKSLGTQNVYDIGVGEPHNFLLKNGLVASN
N-terminus intein domain:
(SEQ ID NO: 19)
CLSYDTEILTVEYGFLPIGKIVEERIECTVYTVDKNGFVYTQPIAQWHN
RGEQEVFEYCLEDGSIIRATKDHKFMTTDGQMLPIDEIFERGLDLKQVD
GLP

The extein, in which eGFP was utilised in this example (+N-terminus 2×Strep tag). Wildtype Cfa splice junctions (CFN & AEY) were incorporated to improve efficiency:

(SEQ ID NO: 20)
CFNWSHPQFEKGGGSGGGSGGSAWSHPQFEKGGSGGEFMVSKGEELFTG
VVPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKT
RAEVKFEGDTLVNRIELKGIDFKEDGNILGHKLEYNYNSHNVYIMADKQ
KNGIKVNFKIRHNIEDGSVQLADHYQQNTPIGDGPVLLPDNHYLSTQSK
LSKDPNEKRDHMVLKERVTAAGITLGMDELYKAEY

The oxygen dependent degradation (ODD) domain from HIF-1α, comprising the key residue P564 for hydroxylation, was incorporated C-terminus to N-terminus intein domain:

(SEQ ID NO: 21)
NPFSTQDTDLDLEMLAPYIPMDDDFQLRSFDQLSPLESSSASPESASPQ
STVTVFQ

The fluorescent protein mCherry was further fused C-terminal to ODD domain, followed by a FLAG tag for antibody recognition:

(SEQ ID NO: 22)
MVSKGEEDNMAIIKEFMRFKVHMEGSVNGHEFEIEGEGEGRPYEGTQTA
KLKVTKGGPLPFAWDILSPQFMYGSKAYVKHPADIPDYLKLSFPEGFKW
ERVMNFEDGGVVTVTQDSSLQDGEFIYKVKLRGTNFPSDGPVMQKKTMG
WEASSERMYPEDGALKGEIKQRLKLKDGGHYDAEVKTTYKAKKPVQLPG
AYNVNIKLDITSHNEDYTIVEQYERAEGRHSTGGMDELYKTGDYKDDDD
K

Two versions of an eGFP extein plasmid (FIG. 4) were generated.

1. Wildtype or WT—will splice and degrade.

2. P564G—mutation of proline 564 in the ODD domain prevents degradation.

### Example 2

The two plasmids from example 1 were independently transfected into Hela cells which were subsequently placed in the presence of oxygen without or with 100 UM of DFX treatment, in order to prevent the degradation of the inteins. In the wildtype plasmid, the inteins should only degrade in the presence of oxygen and be stabilised in the absence of oxygen or in the presence of DFX, due to inhibition of the HIF prolyl hydroxylase domain (PHD). In the P564G mutant, in comparison, the inteins should never degrade, either in the presence of oxygen or with DFX treatment. The proline has been mutated into a glycine, hence preventing the proline from being hydroxylated by PHDs and the subsequent degradation of the protein.

Hela cells were imaged using a Zeiss fluorescent microscope to determine whether the extein (GFP) and the inteins (mCherry-tagged) were present depending upon the conditions.

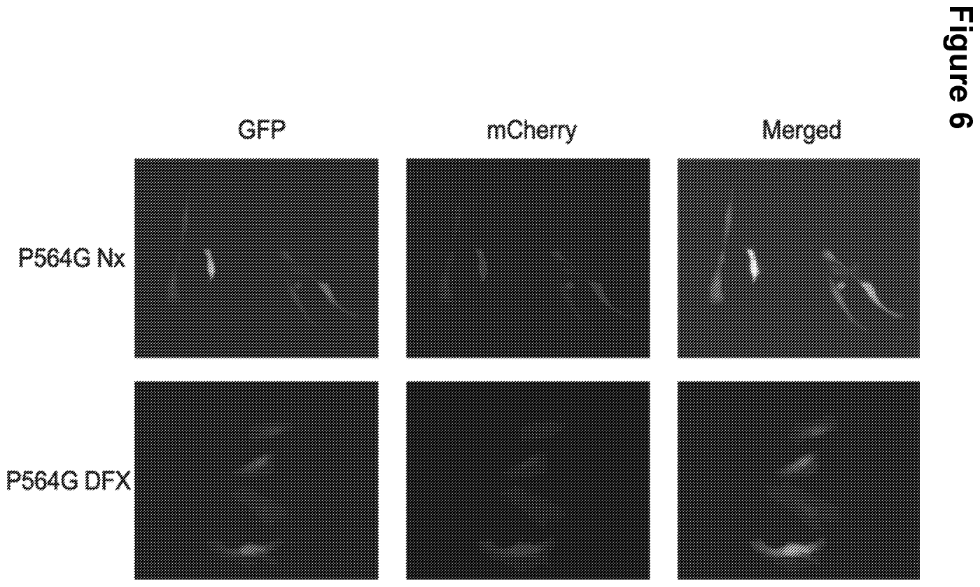

As shown in FIG. 5, it was found that the WT inteins, associated with mCherry fluorescence, degraded in normoxia, in comparison to DFX experiments which displayed mCherry fluorescence. The P564G mutant (FIG. 6) displayed both GFP and mCherry fluorescence in normoxia and DFX, showing that degradation of the inteins does not occur.

### Example 3

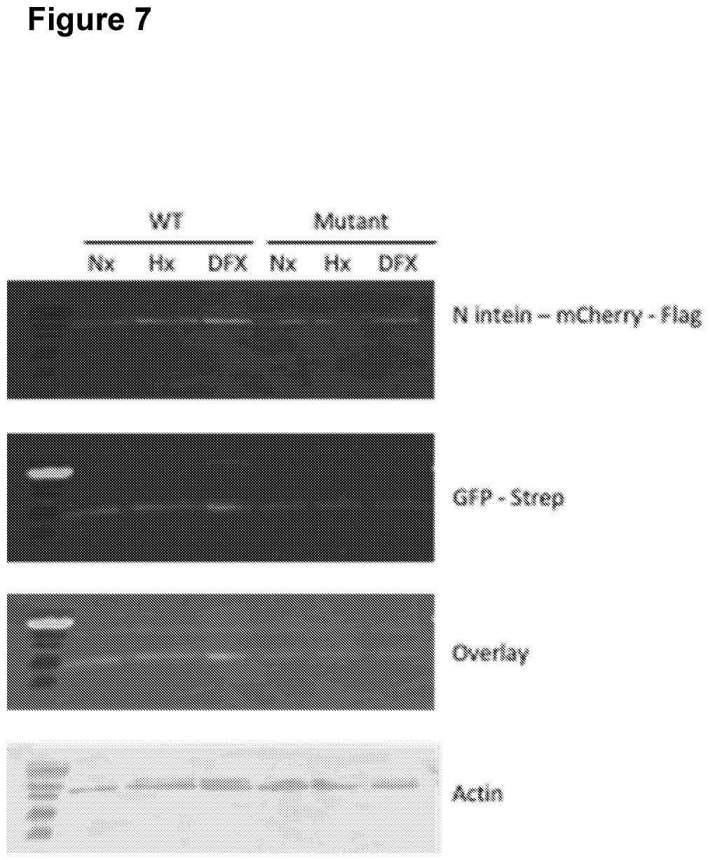

The wildtype and P564G plasmids from examples 1 and 2 were transfected into HeLa cells which were then incubated for 24 hours in normoxia, hypoxia, or DFX-treated conditions. Cells were then lysed using RIPA buffer and scrapers. Total protein lysates were analysed by western blot, the results of which are shown in FIG. 7. Strep tags were used to capture GFP, whilst FLAG tags were used for mCherry. Anti-FLAG and anti-Strep tag antibodies were recognized using secondary antibodies coupled to Alexa 488 and Alexa 568, respectively.

For the wildtype plasmid, hypoxia and DFX conditions displayed more mCherry, showing no degradation of the inteins. Overall, the results show that there was more GFP (associated with the extein) in the WT than P564G mutant, under all conditions. Actin, used as a control, was comparable overall between the wells.

### Example 4

Cell counts were carried out from trypsinised cells that had been independently transfected with the two plasmids from the previous examples, in which the Hela cells had been grown under normoxia and hypoxia conditions. The trypsinised cells were re-suspended in complete media. Cell suspensions were then analysed using a MOXI cell counter to count live and dead cells. Time points were taken at 0h, 6h, 12h, 24h, 36h and 48h in triplicate or duplicate.

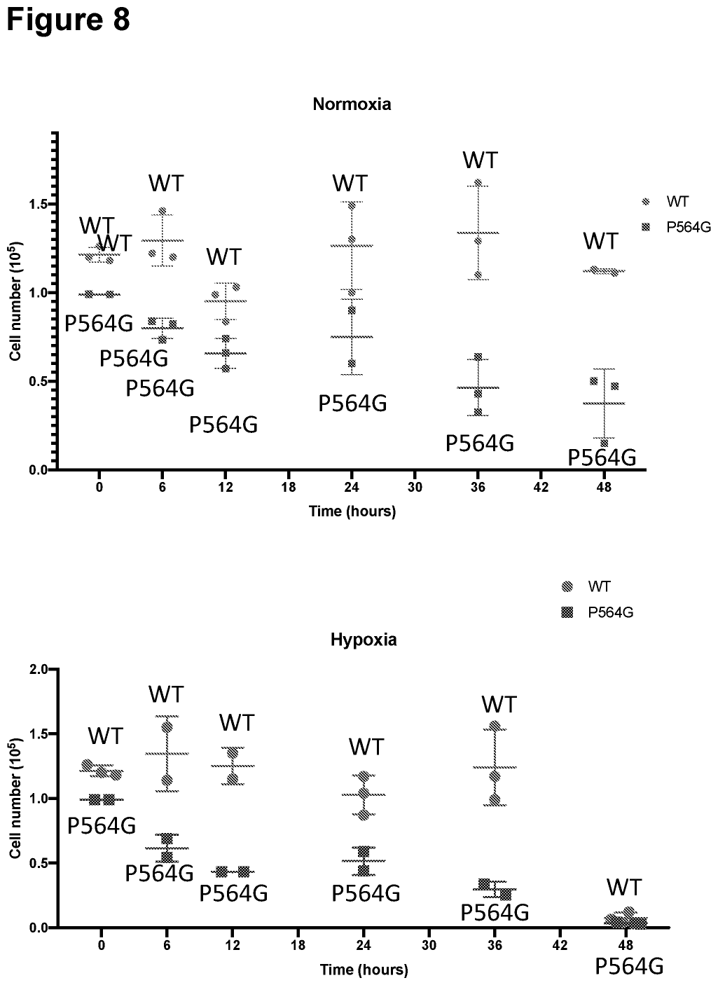

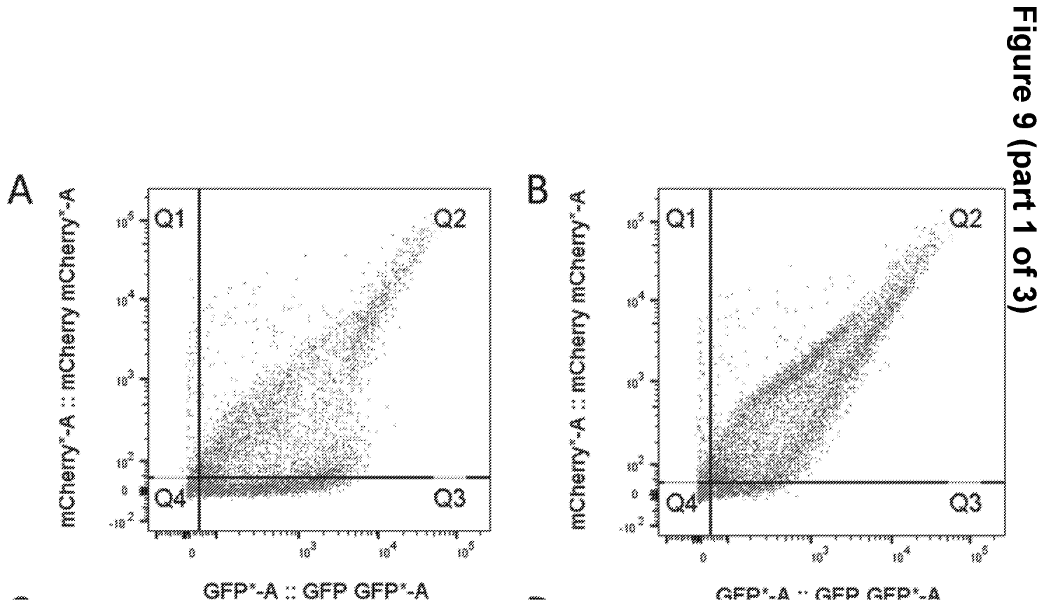

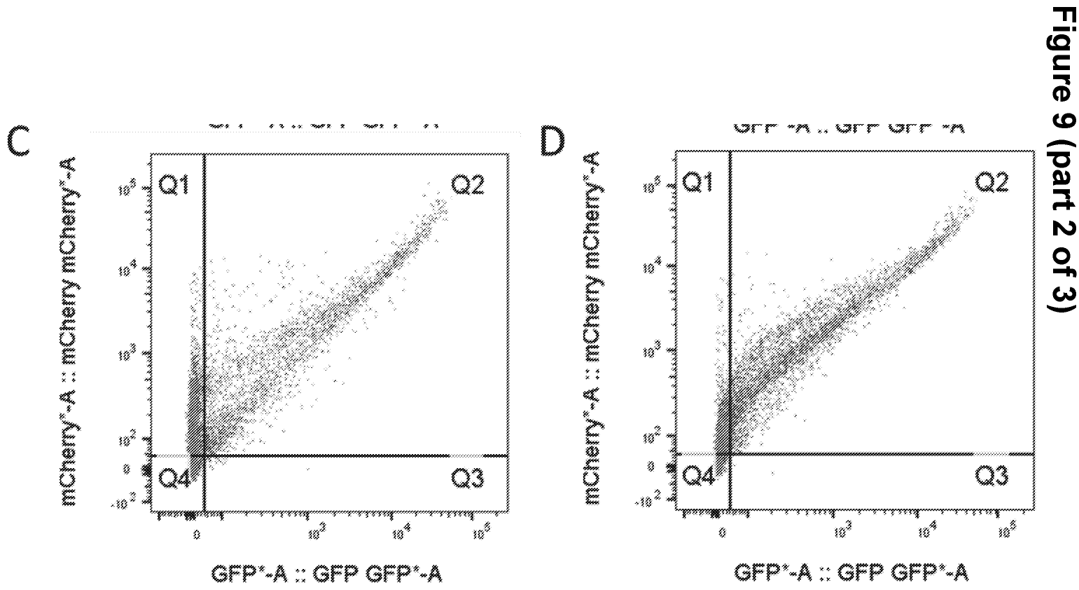

As shown in FIG. 8, the trend shows maintenance of live cell numbers in wildtype-transfected cells, versus a decrease in live cell number in P564G in normoxia. Cells incubated with hypoxia might show a decrease in cell number, i.e. toxicity, from 48h for both plasmids, wherein degradation of the inteins is not occurring.

### Example 5

Transient Transfection of GFP-Npu-mCherry-ODDD Constructs, Splicing (WT) and Non-Splicing (C1A) into Trex Cells

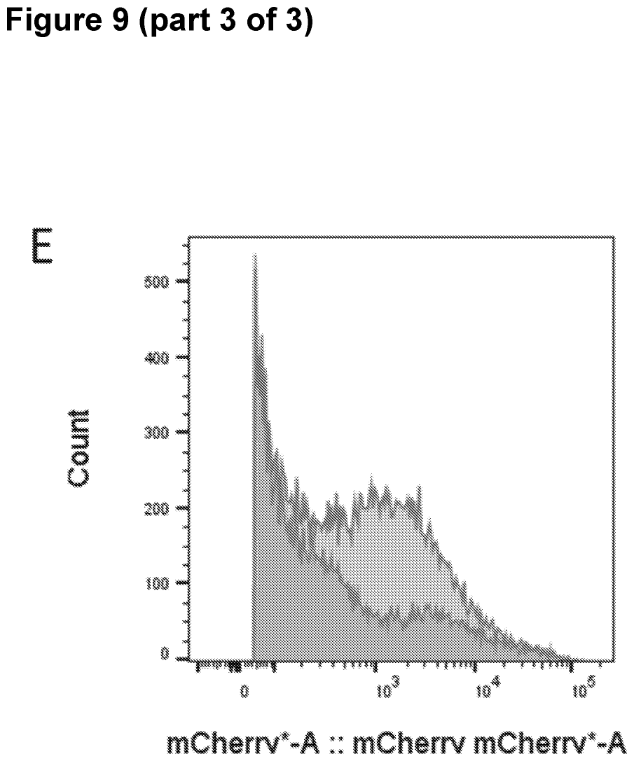

Procedure: 1.2×10⁶ Trex293 cells were plated into 6 cm dishes. Next day, cells were transfected with 1 μg of plasmid and one well remained non-transfected to use as negative control to set fluorescence gates. Next day, media was changed, 1 ug/mL of doxycycline was added to each dish and in the dishes of treated cells, DFX was added to a final concentration of 100 μM. Cells were analysed by FACS on the following day. Results: We investigated whether the inteins fused to mCherry and the Oxygen-Dependent-Degradation-Domain (ODDD) were degrading in presence of oxygen and if addition of DFX could prevent this oxygen-dependent degradation. When cells were analysed by FACS, the GFP splicing version (FIG. 9 panel A) showed cells in the Q3 population (GFP+mCherry-) indicating that the GFP extein spliced and that the inteins alone degraded. Upon addition of DFX (FIG. 9 panel B), a lot less cells were observed in Q3 and more were observed in Q2 (GFP+mCherry+) indicating reduced degradation of the inteins. This was confirmed in the non splicing mutant FIG. 9 (panel C and D). An overlay of the mCherry+ cells from panels A and B (presented in FIG. 9 panel E) confirms that the intensity of mCherry fluorescence is higher in presence of DFX, suggesting less degradation of the inteins via the ODDD pathway.

### Example 6

Stable Integration of GFP-Npu-mCherry-ODDD Construct Splicing (WT) into Trex Cells

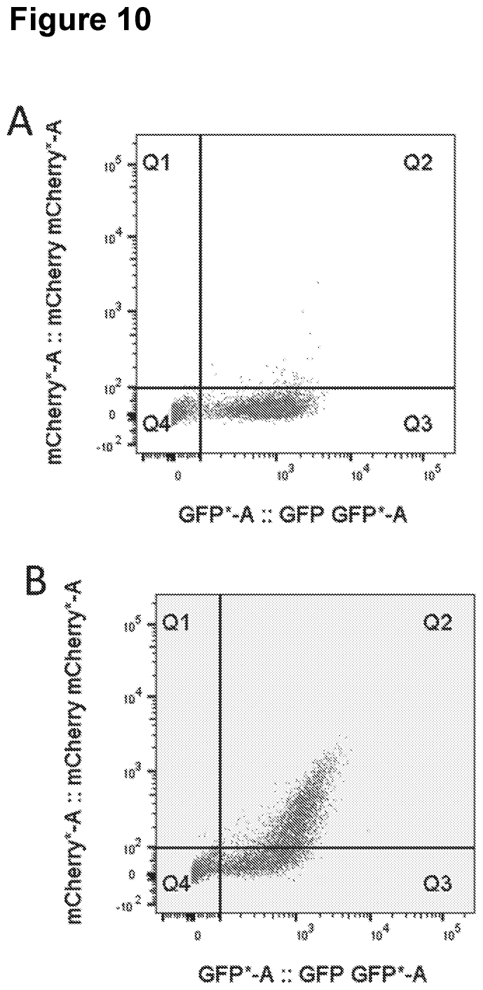

Procedure: Trex293 cells stably integrated with GFP-WT-Npu-mCherry-ODDD were plated in 6-well plates. Cells were then treated with doxycycline to induce the expression of the inteins. One condition was treated with DFX (100 μM) and the other one remained untreated. On the following day, cells were analysed by FACS Results: We investigated whether the inteins fused to mCherry and the Oxygen-Dependent-Degradation-Domain (ODDD) were degrading in presence of oxygen and if addition of DFX could prevent this oxygen-dependent degradation. When the cells were analysed by FACS, in the absence of DFX (FIG. 10 panel A), almost no cells were observed in Q1 and Q2 (mCherry+GFP- and mCherry+GFP+, respectively), with the majority of the cells observed in Q3 (mCherry− GFP+; 67.4%). This indicates that the splicing occurred, separating GFP and mCherry, and that the mCherry-ODDD tagged inteins were degraded in the presence of oxygen, with the spliced GFP remaining intact. Upon addition of DFX (FIG. 10 panel B), many cells were found in Q2 (35.1%), suggesting a decreased degradation of the inteins as more mCherry was present.

### Example 7

Viability Assay of Trex293 Cells Integrated with Constructs Encoding CFA Inteins-Peptide Extein—ODDD—mCherry, Splicing (WT) and Non-Splicing (C1A)

Procedure: Trex cells integrated with CFA inteins—peptide extein—ODDD—mCherry (splicing and non-splicing) were plated at a density of 1000 cells per well in 96-well plates. Next day, media was changed and replaced with fresh media containing doxycycline (1 μg/mL) with or without DFX (100 μM final concentration). Next day, cell viability was measured using Cell Titer Glo Assay.

| | Peptide extein | Peptide extein |
|---|---|---|
| | WT | C1A |
| −dfx | 100 | 100 |
| +dfx | 57.22013278 | 57.73246861 |

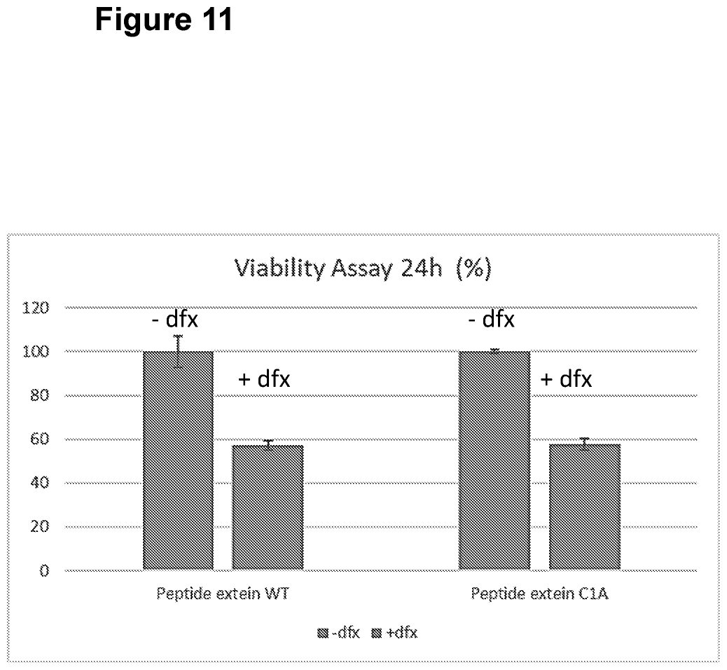

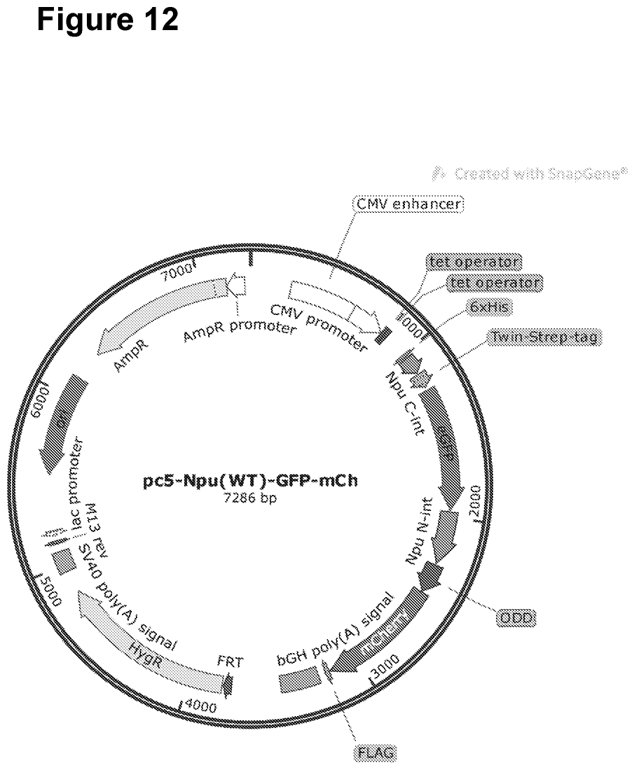

Results: This viability assay confirms that upon treatment with dfx, shown to result in a decrease in the degradation of the inteins, cell viability significantly decreases (FIG. 11). This suggests that the presence of the inteins in the cell has a negative effect upon cell viability. The −dfx control results in the degradation of the inteins and subsequently increased viability. The C1A non-splicing control confirms that this is not a result on extein toxicity, as no spliced extein is present.

Sequences used throughout the specification and forming part of the description:

(Amino acid sequence for Ssp DnaE C-terminus intein domain)
SEQ ID NO: 1
MVKVIGRRSLGVQRIFDIGLPQDHNFLLANGAIAANC
(Amino acid sequence for Ssp DnaE N-terminus intein domain)
SEQ ID NO: 2
AEYCLSFGTEILTVEYGPLPIGKIVSEEINCSVYSVDPEGRVYTQAIAQWHDRGE
QEVLEYELEDGSVIRATSDHRFLTTDYQLLAIEEIFARQLDLLTLENIKQTEEALD
NHRLPFPLLDAGTIK
(Amino acid sequence for Npu DnaE C-terminus intein domain)
SEQ ID NO: 3
MIKIATRKYLGKQNVYDIGVERDHNFALKNGFIASN
(Amino acid sequence for Npu DnaE N-terminus intein domain)
SEQ ID NO: 4
CLSYETEILTVEYGLLPIGKIVEKRIECTVYSVDNNGNIYTQPVAQWHDRGEQEV
FEYCLEDGSLIRATKDHKFMTVDGQMLPIDEIFERELDLMRVDNLPN
(Amino acid sequence for Cfa DnaE C-terminus intein domain)
SEQ ID NO: 5
VKIISRKSLGTQNVYDIGVEKDHNFLLKNGLVASN
(Amino acid sequence for Cfa DnaE N-terminus intein domain)
SEQ ID NO: 6
CLSYDTEILTVEYGFLPIGKIVEERIECTVYTVDKNGFVYTQPIAQWHNRGEQEV
FEYCLEDGSIRATKDHKFMTTDGQMLPIDEIFERGLDLKQVDGLP
(Amino acid sequence for gp41-1 C-terminus intein domain)
SEQ ID NO: 7
MMLKKILKIEELDERELIDIEVSGNHLFYANDILTHNS
(Amino acid sequence for gp41-1 N-terminus intein domain)
SEQ ID NO: 8
CLDLKTQVQTPQGMKEISNIQVGDLVLSNTGYNEVLNVFPKSKKKSYKITLEDG
KEIICSEEHLFPTQTGEMNISGGLKEGMCLYVKE
(Amino acid sequence for Homo sapiens hypoxia-inducible factor-1 alpha
[HIF-1α] subunit)
SEQ ID NO: 9
MEGAGGANDKKKISSERRKEKSRDAARSRRSKESEVFYELAHQLPLPHNVSS
HLDKASVMRLTISYLRVRKLLDAGDLDIEDDMKAQMNCFYLKALDGFVMVLTDD
GDMIYISDNVNKYMGLTQFELTGHSVFDFTHPCDHEEMREMLTHRNGLVKKGK
EQNTQRSFFLRMKCTLTSRGRTMNIKSATWKVLHCTGHIHVYDTNSNQPQCG
YKKPPMTCLVLICEPIPHPSNIEIPLDSKTFLSRHSLDMKFSYCDERITELMGYEP
EELLGRSIYEYYHALDSDHLTKTHHDMFTKGQVTTGQYRMLAKRGGYVWVET
QATVIYNTKNSQPQCIVCVNYVVSGIIQHDLIFSLQQTECVLKPVESSDMKMTQL
FTKVESEDTSSLFDKLKKEPDALTLLAPAAGDTIISLDFGSNDTETDDQQLEEVP
LYNDVMLPSPNEKLQNINLAMSPLPTAETPKPLRSSADPALNQEVALKLEPNPE
SLELSFTMPQIQDQTPSPSDGSTRQSSPEPNSPSEYCFYVDSDMVNEFKLELV
EKLFAEDTEAKNPFSTQDTDLDLEMLAPYIPMDDDFQLRSFDQLSPLESSSASP
ESASPQSTVTVFQQTQIQEPTANATTTTATTDELKTVTKDRMEDIKILIASPSPTH
IHKETTSATSSPYRDTQSRTASPNRAGKGVIEQTEKSHPRSPNVLSVALSQRTT
VPEEELNPKILALQNAQRKRKMEHDGSLFQAVGIGTLLQQPDDHAATTSLSWK
RVKGCKSSEQNGMEQKTIILIPSDLACRLLGQSMDESGLPQLTSYDCEVNAPIQ
GSRNLLQGEELLRALDQVN
(Amino acid sequence for the oxygen dependent degradation (ODD) domain
of Homo sapiens hypoxia-inducible factor-1 alpha [HIF-1α] subunit)
SEQ ID NO: 10
NPFSTQDTDLDLEMLAPYIPMDDDFQLRSFDQLSPLESSSASPESASPQSTVT
VFQ

1. A method for the non-toxic production of a cyclic peptide in a mammalian cell comprising:
a) introducing a vector into the mammalian cell, wherein the vector comprises a construct encoding a C-terminus intein domain and a N-terminus intein domain of a split intein;
a polypeptide sequence to be cyclised; and a degradation tag, wherein the degradation tag is attached to at least one intein domain;
b) expressing the construct to produce an intermediate comprising an active intein and the polypeptide sequence, whereby the active intein undergoes splicing and cyclises the polypeptide and wherein the degradation tag degrades the active intein.

2. A cyclic peptide library produced by the method according to claim 1.

3. A mammalian cell expressing a cyclic peptide wherein the mammalian cell is produced by a method comprising:
a) introducing a vector into the mammalian cell, wherein the vector comprises a construct encoding a C-terminus intein domain and a N-terminus intein domain of a split intein;
a polypeptide sequence to be cyclised; and a degradation tag, wherein the degradation tag is attached to at least one intein domain;
b) expressing the construct to produce an intermediate comprising an active intein and the polypeptide sequence, whereby the active intein undergoes splicing and cyclises the polypeptide and wherein the degradation tag degrades the active intein.

4. The mammalian cell according to claim 3 wherein the cell comprises no active inteins or substantially no active inteins.

5. A genetic construct comprising: a polynucleotide cassette encoding a C-terminus intein domain and a N-terminus intein domain of a split intein; a polypeptide sequence to be cyclised; and a degradation tag suitable for use in mammalian cells, wherein the degradation tag is attached to at least one intein domain.

6. A vector comprising the genetic construct according to claim 5.

7. A mammalian cell comprising the genetic construct according to claim 5 and/or the vector according to claim 6.

8. The method, library, construct, vector or cell according to any preceding claim 1, wherein the active intein splices before it is degraded by the degradation tag.

9. The method, library, construct, vector or cell according to any preceding claim 1, wherein the degradation tag effects degradation at least in part by ubiquitination.

10. The method, library, construct, vector or cell according to any preceding claim 1, wherein the degradation tag is the hypoxia-inducible factor-1 alpha (HIF-1α) subunit or a proteolysis targeting chimera (PROTAC).

11. The method, library, construct, vector or cell according to any preceding claim 1, wherein the degradation tag is the oxygen dependent degradation (ODD) domain of the hypoxia-inducible factor-1 alpha (HIF-1α) subunit comprising the key residue P564.

12. The method, library, construct, vector or cell according to claim 11, wherein the ODD domain of HIF-1α comprises the sequence length spanning amino acids 548-603.

13. The method, library, construct, vector or cell according to any of claims 1-9 claim 1, wherein the degradation tag is a proteolysis targeting chimera (PROTAC) small molecule capable of engaging an E3 ubiquitin ligase.

14. The method, library, construct, vector or cell according to any preceding claim 1, wherein the active intein is a Cfa, Npu, Ssp or gp41-1 intein.

15. The method, library, construct, vector or cell according to any of claims 1-13, wherein the active intein is an Npu intein.

16. The method, library, construct, vector or cell according to any preceding claim 1, wherein the linkage between the degradation tag and the intein is a direct linkage.

17. The method, library, construct, vector or cell according to any preceding claim 1, wherein the construct further encodes at least one affinity tag.

18. The method, library, construct, vector or cell according to claim 16, wherein the at least one affinity tag encoded is a FLAG-tag for antibody recognition.

19. The method, library, construct, vector or cell according to any preceding claim, wherein the construct further encodes a fluorescent tag, wherein said tag is preferably DsRed.

20. A method of producing a cyclic library using the method of claim 1.

21. The method, library, construct, vector or cell according to any preceding claim 1 wherein the intein is toxic to mammalian cells.
